(19) United States
(12) Patent Application Publication
(10) Pub. No.: US 2011/0046687 A1
(43) Pub. Date: Feb. 24, 2011

(54) LIVE COMBINED STIMULATION OF AUDITORY, SENSORY AND MOTOR FUNCTIONS AND ENHANCED THERAPEUTIC AND COMMUNICATIVE APPLICATIONS BASED ON ADVANCED GENERATION OF COMPLEX ELECTRICAL SIGNALS

(76) Inventor: Raimund Naschberger, Aldrans (AT)

Correspondence Address: Raimund Naschberger, Grubenweg 5b, Aldrans, 6071, omitted

(21) Appl. No.: 12/856,582

(22) Filed: Aug. 13, 2010

Related U.S. Application Data
(60) Provisional application No. 61/277,772, filed on Aug. 14, 2009.

(51) Int. Cl. A61N 1/02

(52) U.S. Cl. 607/3

(57) ABSTRACT

A stimulator device is disclosed that is capable to produce and apply synchronized complex electrical stimulation signals very responsive and user friendly with interactive ability to change and derivate multiple signals or processing parameters immediately and simultaneously. The stimulator processes signals e.g. music and is typically controlled in real-time by a communication protocol e.g MIDI data or live by a MIDI controller. Immediate patient response and adoption facilitates the development of more efficient, diversified and enjoyable stimulation content for the majority of therapies based on electrical activated transducers. Special scope is combined auditory, electrical and vibration stimulation of the sensory and motor functions. One disclosed embodiment is enhanced audio perception for deaf and hearing impaired patient. Another typical application is combined music, electrical and vibration stimulation e.g. for neck and back muscle relaxation or thrombosis prophylaxis provided by a PC based stimulation content generator and a protable player.

This application claims priority from U.S. Provisional Patent 61/277,772, filed Aug. 14, 2009, which is incorporated herein by reference.

### TECHNICAL FIELD

The present invention relates to therapeutic use to cure or alleviate health conditions by use of electrical stimulation accompanied with a nonelectrical therapy and more specifically to the generation of complex electrical signals to drive a combined set of transducers for auditory, sensory and motor functions.

### BACKGROUND ART

Auditory stimulation is widely used and known for a long time starting by natural hearing of music and speech up to listening to music and speech reproductions by speakers and headphones. Vibrational representation of sound, typically bass is applied by natural musical instruments, sound pressure of speakers or vibrations evoked by shakers. Such applications are common for entertainment e.g. cinema and multimedia but also for therapies e.g. singing bowls. For mild to severe hearing impaired individuals hearing instruments based on acoustic amplification of sound are widely used. For severe to profound hearing impaired patient's middle ear implants and bone conduction implants based on vibratory stimulation and cochlea implants based on electrical stimulation are established state of the art therapies. Recently a combination treatment provided by a cochlea implant with integrated but separately processed hearing instrument proves significant better speech recognition and music perception for specific indications.

Stimulation of motor function is well established for a long time starting with electrotherapy for the regeneration and training of all kind of muscles e.g. back- neck-, extremities up to research for gait stimulation to assist paraplegic patients. Many wellness and slender applications are based on stimulation of motor functions. The majority of applications use electrical stimulation either by implanted or surface electrodes. It is quite popular to stimulate muscles by surface electrodes that adhere by suction. In some embodiments low frequency modulation of the suction vacuum adds a comfortable massage effect to the electrical stimulation. In addition many mechanical apparatuses for vibrational massages are know for therapy, sports- and wellness.

Stimulation of sensory motor function is as well well established for a long period in particular for pain reduction and masking either by transcutaneous (TENS) or implantable devices e.g. for the treatment or epilepsy, Parkinson and other nervous disorders. In addition devices for microcurrent stimulation therapies and electroacupuncture are common available.

Signal generation for electrical stimulators for the stimulation of the sensory is commonly implemented with instruction based programming. Stimulation signals are generated by computer instruction that typically activate timers and I/O ports that define the stimulation signal. The programs operate with interrupts and polling loops. Such the generators are practically limited in their flexibility to generate and output multiple signals synchronously with stable defined timing. The shape of selectable pulses and pulse trains are limited, typically restricted to bipolar pulses.

Other applications perform playback of stored waveforms at predefined or adjustable sample rates. The shape of selectable waveforms is limited. With both principles rapid and flexible changes of multiple signal parameters or seamless changes of the shape of the signals are a real challenge or practically not possible.

Today's treatment schemes are static to a high degree. Typically stimulation level change linear and predefined pulse trains change in switched mode over several minute periods. 1^{st} and 2^{nd} generation medical treatments could be much easier investigated and clinically approved with such little change. Hearing instruments and implants perform single stimulation, acoustical, vibrational or electrical. Acoustical and vibrational systems directly apply signal conditioned sound signals. Electrical systems in particular cochlea implants derivate from a sound signal a fixed electrical pulse stimulation scheme. While a cochlea implant, representative for the state of the art of a communication stimulator, changes sufficient rapidly stimulation parameters, the processing is based on limited and fixed preconfigured stimulation strategies for electrical stimulation only.

All such forementioned technical solutions are highly depended on the specific hardware involved and such proprietary, preconfigured and highly specialized for specific applications. Stimulation and therapy schemes, once they are established remain permanent hardwired or are subject to instructional data where update and variation of functions is limited.

### SUMMARY OF INVENTION

The disclosed physical therapy apparatus is capable to stimulate the external auditory pathways, muscles, nerves and tissue including reflex points in the body simultaneously and synchronised. The stimulator device facilitates a flexible course of combined and synchronized multichannel multitype stimulation and applies complex electrical signals that drive transducers for the stimulation of the auditory, sensory and motor functions. The stimulator is capable to apply electrical, vibratory or both stimulations and apply in addition an audio signal or derivate electrical or vibratory stimulation from an audio signal. While transducers are typically producing acoustic waves, mechanical vibrations or variations of electric current or voltage the invention does not disclose novel transducers.

The operation of the generator can be controlled for immediate or subsequent delivery and change of the parameters of a desired stimulation signal. Digital signal processing of data streams- or protocols, consistent for all kind of stimulation, enable a flexible course of combined and synchronized multichannel and multimode stimulation and emerge established quasi static stimulation and single type stimulation therapies. The invention overcomes the lack of flexibilty of instruction stream based computing architecture based on van Neumann machine.

The stimulator facilitates the generation of a virtually unrestricted repertoire of waveforms and sequences either by algorithms or stored sampled waveform templates. Processing of multichannel and multitype applications are facilitated and supported by a consistent data format and protocol. In addition multiple signal- and processing parameters can be changed and modulated immediately and very responsive. Input signals can be incorporated and function as feedback and control signals. Where beneficial e.g. for research and development of new applications, a modular concept with flexible routing and mixing of functional modules e.g. pulse generators, envelopes, oscillators, signal processing and modulation modules accomplish very flexible signal processing and functions. The modules are capable to interface with multimedia applications by SW plug-in standards. Optional the stimulator device can sample, process and continue to use signals and therapies of existing hardware stimulators.

The stimulator is typically controlled in real-time by a communication protocol. Data words thereof are sufficiently comprehensive to describe, control and synchronize the stimulation and the repetition is sufficiently high to modify signals for the targeted application. Preferred is the musical instrument digital interface MIDI. A specialized controller e.g. with keys, pads, knobs, sliders preferable a MIDI controller facilitates very user friendly and responsive the generation of stimulation programs. The stimulator may be operated live, even with remote data transfer over long distance and provide immediate patient feedback and such more efficient, diversified and enjoyable stimulation content for the majority of therapies is facilitated. This is done much more efficiently and in a fraction of time needed to re-program or even reconstruct instruction based stimulators.

The invention supports and encourages the generation and playback of a diversified collection of combined stimulation content. While stimulation content is typically generated on a specialized PC production environment, a separate portable player loads or streams stimulation data. In another embodiment the player function is a SW-application of a standard mobile phone that drives a physical stimulation interface. This facilitates effective distribution of stimulation programs and application.

The disclosed invention facilitates novel and improves existing applications for human and animal necessities for in particular health and facilitates a better reception for a majority of medical, sports and wellness applications. General scope is combined auditory, electrical and vibration stimulation of the sensory and motor functions. A typical application is to apply live and enjoyable combined music, electrical and vibration stimulation. Neck, back and muscle relaxation as well as thrombosis prophylaxis are primary target applications. Immediate patient response and adoption facilitates the development of more efficient, diversified and enjoyable stimulation content for the majority of therapies based on electrical activated transducers. While primary scope is therapeutic use to cure or alleviate health conditions well being and ambient living applications are as well facilitated or potentially improved by the invention.

In addition the disclosed implementations leverage the stimulator to a communication instrument with the potential to generate a meaningful representative supplement or substitute of the auditory perception by other senses evoked by vibrational and electrical stimulation. Specific scope of the invention is to derive electrical and vibration stimulation signals from an audio source and mimic characteristic properties and effects of music and speech to facilitate and enhance audio perception for deaf and hearing impaired individuals.

### BRIEF DESCRIPTION OF DRAWINGS

FIG. 1 shows the essential functional modules of the minimum basic stimulator device and how they are related and interact with each other.

FIG. 2 shows an embodiment of FIG. 1 with separate housings for the generator and the player functions.

FIG. 3 illustrates a typical embodiment of FIG. 2 with a PC production setup and MIDI controller and an external stimulator player hosted by a touchscreen mobile phone.

FIG. 4 shows the functional modules of the modular scheme of the physical stimulation generator module. In FIG. 1-FIG. 4 1 shows a stimulation data link, 2 shows a general data link, 3 shows a auditory stimulation applicator, 4 shows a vibratory stimulation applicator, 5 shows a electrical stimulation applicator, 6 shows a human or animal body, 7 shows a controller, typically a MIDI instrument type controller, 8 shows a physical stimulation generator, 9 shows a physical stimulation player, 10 shows a physical stimulation interface, 11 shows a computer system, typically a PC, portable multimedia or telecommunication device, 12 shows a housing, 13 shows a control signal, typically related to the stimulation, 14 shows an oscillator module, 15 shows an envelope module, 16 shows a routing and mixer module, 17 shows a digital signal processing module related to the stimulation, 18 shows a derivation module, 19 shows a safety module, 20 shows a general input signal, typically related to the stimulation, 21 shows an internal data link related to the stimulation.

FIG. 5 shows as the graphical user interface of a 2-channel universal stimulator based on the modular scheme shown in FIG. 4.

FIG. 6 shows a train of biphasic stimulation pulses with levels following the envelope.

FIG. 7 shows a typical setup of a portable combined multimedia and stimulation player.

FIG. 8 shows a block diagram of a typical physical stimulation interface (Ph-Stim).

FIG. 9 shows a block diagram of a typical physical input interface (Ph-In).

FIG. 10 shows an application specific embodiment for combined music, electrical- and vibrational stimulation.

FIG. 11. shows a virtual physical stimulator device that samples, mimics and emulates an existing hardware stimulator.

### DESCRIPTION OF EMBODIMENTS

Various embodiments of the present invention are directed to a stimulator device for the stimulation of a combination of auditory, sensory and motor functions. Typically the stimulation of the sensory and motor functions is derived from an audio signal or the audio signal changes and modulates the stimulation signal for the sensory and motor functions. This is put into practice by implementing the overall concept described in the summary of the invention, in particular digital signal processing of data streams or communication protocols representing the stimulation. The method comprises digital signal processing at least for the generation of complex electrical stimulation waveforms with interactive ability to change multiple processing parameters immediately and simultaneously, a communication protocol that enables at least the physical stimulation generator module to communicate control and synchronize the processing of stimulation data. Preferably a musical instrument type controller controls at least the physical stimulation generator module by the communication protocol. Preferably an advanced graphical user interface immediately displays function, operation and configuration parameters such that a virtual representation of a hardware embodied device is given. Mandatory for a medical device, user safety monitoring and limiter function are implemented to comply with at least with regulatory requirements. Preferable such limits are configurable by a protected method and configured to meet user comfortable levies. While it is apparent for those skilled in the art that various methods lead to the design and manufacture of the disclosed embodiments a preferred implementation is described as follows:

The invention is build up of functional modules and submodules within a flexible modular concept. Each functional module can be implemented either as hardware or software or a combination thereof. Each functional module represents in hardware an integrated circuit e.g. ASIC with a defined electrical function e.g. an oscillator and pin-out of electrical I/O signals. Such signals are either preconfigured or flexibly patched to other functional modules building the whole stimulator system. In software the functional modules with pin-out are emulated by algorithms, typically embodied in a software procedure that is an element of the whole digital signal processing program. The routing of the signals is modelled by the program flow and the data exchange between software routines. Typically a graphical user interface performs a virtual representation of the stimulator hardware and allows for flexible editing and patches. Such visual programming and modelling is typically performed in a hardware description language HDL. The whole digital signal processing and overall function of the major modules of the stimulator is performed in realtime by simulation software. Such simulation software exchanges data with the hosting computer system and performs the virtual representation of the stimulator device. Data exchange with other software applications is achieved by common data link technologies. Preferably plug-in technologies and dynamic link libraries are utilized. The stimulator integrates with multimedia applications by plug-ins like VST and Rewire and utilizes signals, data and functions provided form the operating system OS or other software applications. Preferably the software embodiments and data formats of the stimulator modules are specified and designed to allow the usage of existing I/O hardware and communication means and portability to different computer systems either for PC's or portable multimedia and communication devices. Computer systems in this context and drawings is understood as a combination of computer hardware including I/O devices, human interface, display, data transfer links independently how processing is performed e.g. by a CPU, microcontroller, DSP or a combination thereof, and computer software or firmware including an optional operating system and other system or application software.

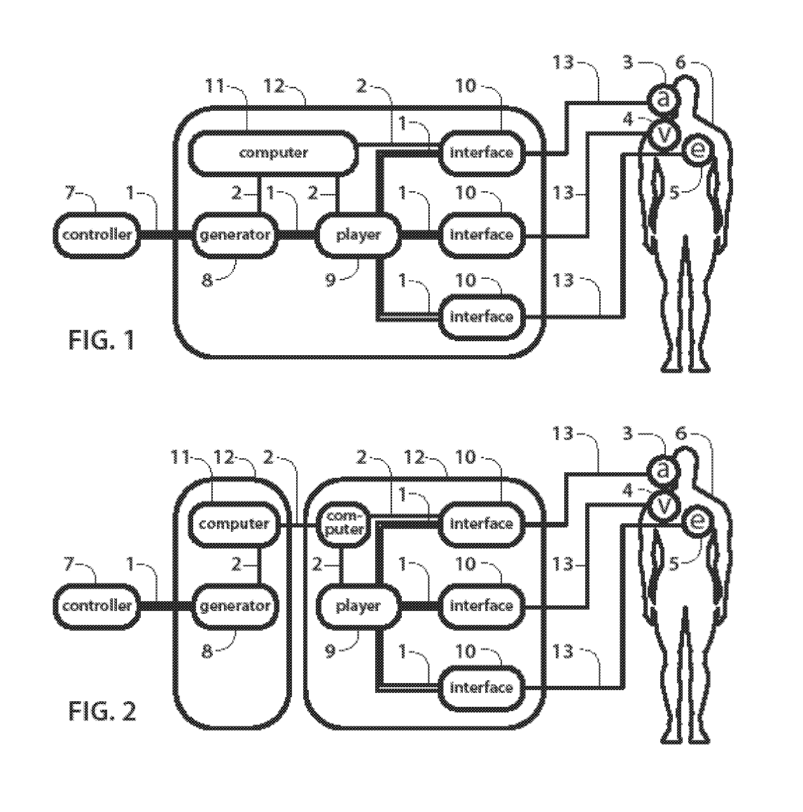

FIG. 1 shows the essential functional modules of the minimum basic stimulator device and how they are related and interact with each other. The core device is build up by a physical stimulation generator 8, a player 9, at least one or more separate embodied physical stimulation interfaces 10 that drive by control signals 13 either at least one auditory stimulation transducer 3, at least one vibratory stimulation transducer 4, at least one electrical stimulation transducer 5 or a combination thereof. The control signal 13 represents the stimulation either as analog or digital signal and can by either an electrical, optical or radio link. Typically a controller 7, preferably a musical instrument type MIDI controller is either directly connected to the generator 8 or routed through the computer system 11 and sends control data representing or defining the stimulation 1. The generator transmits the stimulation data 1 either directly or routed through the computer system 11 online or offline to the player 9 and optional exchanges data with a computer system 11 by a data link 2. Such data may comprise the storage and retrieval of stimulation data, control data including MIDI, data representing input and output signals, in particular audio and control signals and other data. In another embodiment above mentioned data link 2 is directly established from the outside environment to the generator. The player 9 transmits the stimulation data 1 either directly or routed through the computer system to the physical stimulation interfaces 10. The player 9 optional exchanges data with a computer system 11 by a data link 2. Such data may comprise the storage and retrieval of stimulation data, control data including MIDI, data representing input and output signals, in particular audio and control signals and other data. In another embodiment above mentioned data link 2 is directly established from the outside environment to the player.

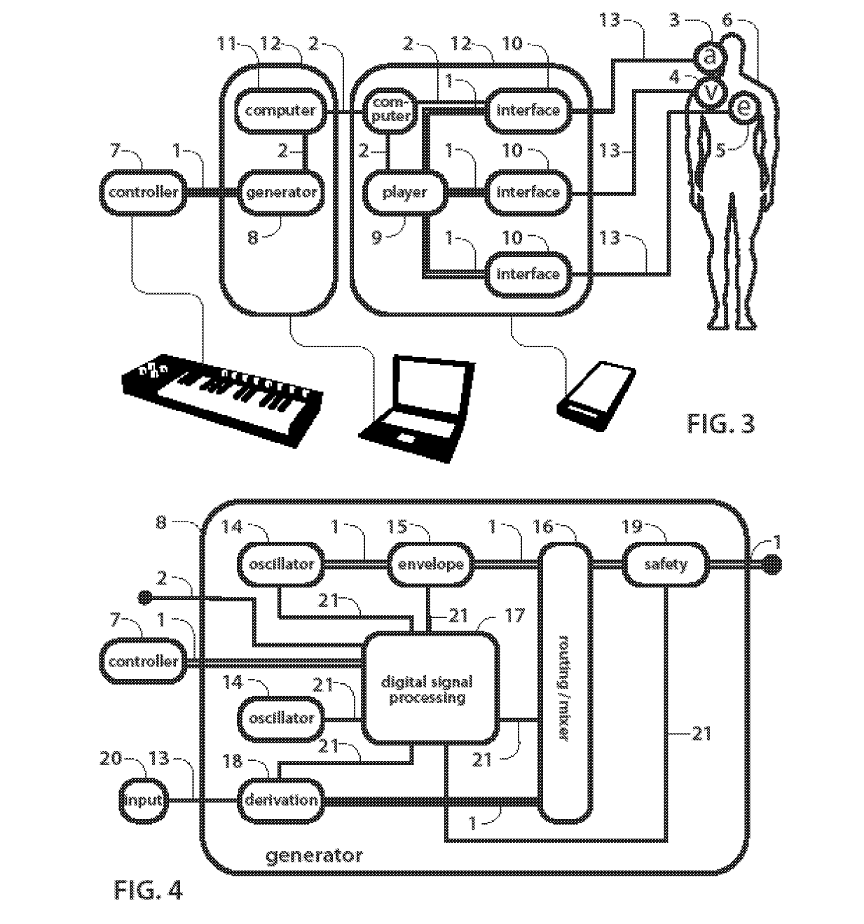

Typical embodiments of FIG. 1 for the housing 12 are personal computers, game consoles, mutimedia receivers and setup boxes. In addition multimedia and telecommunication devices, preferable portable embodiments thereof, may contain the functions of FIG. 1. In such an embodiment typically the controller 7 and at least the physical connector of the stimulation data link 1 thereof, e.g. MIDI is not implemented and substituted by other integrated or external human interface devices through the computer system. FIG. 2 shows an embodiment of FIG. 1 with separate housings 12 for the generator 8 and the player 9 functions. In such embodiment the generator is typically implemented in a personal computer which functions as a production platform. Preferably the personal computer is equipped with hardware and software for multimedia production. The player 9 is typically implemented in a portable multimedia or telecommunication device and typically is connected by a data link 2 to the separate computer system 11 of such device. Audio stimulation is typically provided directly by such computer system and the physical stimulation interface for the auditory stimulation is an integrated audio interface. The physical stimulation interface for the vibratory and electrical stimulation is either integrated but preferably an external device. Such embodiment facilitates the usage of standard commercially available computer, multimedia or telecom devices e.g. a touchscreen mobile phone. Adding a software embodiment of the invention and at least one external physical stimulation interface leverages such device to a stimulator device and allows for integration of combined vibratory and electrical stimulation. FIG. 3 illustrates a typical embodiment of FIG. 2 with a PC production setup and MIDI controller and an external stimulator player hosted by a touchscreen mobile phone.

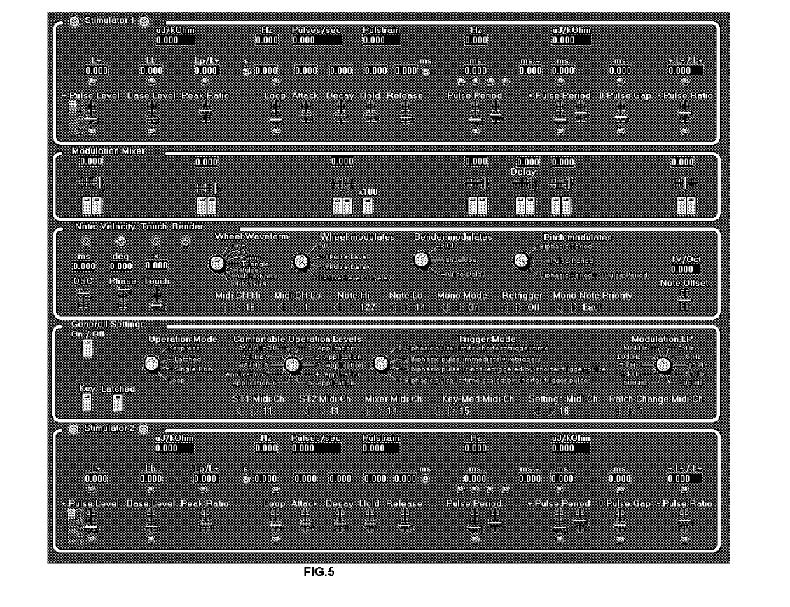

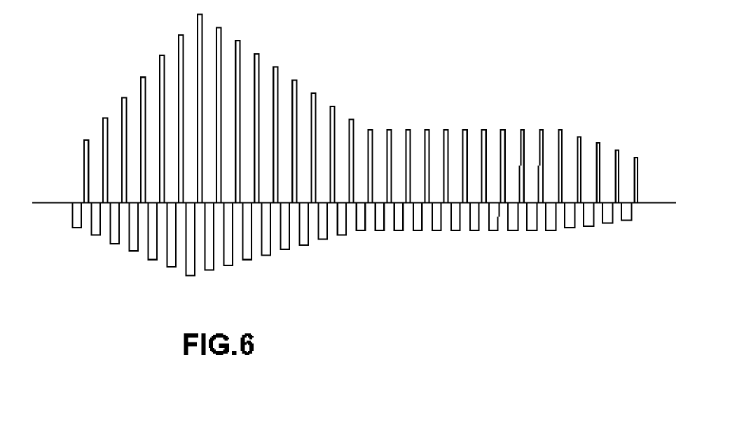

FIG. 4 shows the functional modules and modular scheme of the minimum physical stimulation generator module. The core module is build up by at least one oscillator module 14 that sends stimulation data to at least one envelope module 15 that sends stimulation data to a routing/mixer module 16. The routing/mixer module in a minimum configuration at least outputs the stimulation data either directly or preferably through a safety module 19. In a preferred embodiment an input signal 20 is routed to the derivation module. Optional the input signal 20 is either directly routed through the routing/mixer 16 to the derivation module 18 or indirectly through the digital signal processing module. The derivation module 18 outputs stimulation data through the routing/mixer module 16 and optional receives stimulation data thereof. In a typical embodiment a digital signal processing module 17 provides data links controlling or representing the stimulation 21 to the above mentioned modules, exchanges stimulation data with a controller 7 e.g. MIDI or other external module and provides a data link 2 to a computer system. Optional an oscillator module 14, different from the stimulation oscillator provides modulation of the stimulation signal through the digital signal processing module or directly by the routing/mixer module.

It is understood that all modules and submodules may be applied multiple and with modified functions and algorithms as part of a modular concept. The output stimulation data is typically a multimode and multitype signal. The routing/mixer module 16 is capable to add more than one signal to generate a single stimulation signal. The digital signal processing module itself is typically build from multiple function modules. Stimulation data 1 is either the data representing the stimulation itself or a communication protocol controlling or defining the stimulation at least partially. A data link 2 may comprise any data including storage and retrieval of stimulation data, control data including MIDI, data representing input and output signals, in particular audio and control signals and other data. A control signal 13 represents the stimulation either as analog or digital signal, typically multiple signals and can by either an electrical, optical or radio link.

The physical stimulation generator includes at least one oscillator module that generates biphasic pulses with defined levels, periods and repetition rate. The anodic pulse typically defines the cathodic pulse such that no DC component remains. One parameter of the cathodic pulse e,g, the pulse level in relation to the anaodic pulse level remains configurable. Such biphasic pulse oscillator may be routed to two or more stimulation output with adjustable time delay or in another embodiment separate, preferable identical oscillators for each stimulation output are controlled by the digital signal processing module. In principle each parameter can be changed in realtime e.g. by a communication protocol e.g. in a research embodiment but typically parameters are preselected and limited according to the target application. The routing/mixer module connects the selected modulation signals to the selected parameters. The mixer function sets the gain. The oscillator module can by usually retriggered by the communication protocol or control signal to generated synchronized stimulation signals.

The physical stimulation generator includes at least one oscillator module that generates waveforms based on algorithms including sine, triangle, trapezoid, pulse, triangle, ramp, white- and pink noise. Levels, period, symmetry and phase where applicable are preconfigured and typically a selection thereof is adjustable and controlled e.g. a communication protocol. Typically the modulation wheel of a keyboard controller sets the period of such oscillator. Such oscillator is typically utilized to modulate the parameters of the electrical stimulation signal or to generate vibrational stimulation signals. The oscillator module can by usually retriggered by the communication protocol or control signal to generate synchronized stimulation signals.

The physical stimulation generator includes at least one sample player module that generates waveforms represented by data samples with defined period and repetition rate. Typically the repetition rate, if applied as loop oscillator, is adjustable and controlled by a communication protocol. Typically the waveplayer can select from many stored waveforms and recall multiple waveforms simultaneously and distribute it to one or multiple stimulation channels. Such any waveform and combination thereof can by established. In one embodiment auditory sensations are stored as waveforms. In another embodiment stimulation signals from other sampled stimulation devices are re-used. The sample player can by usually retriggered by the communication protocol or control signal to generate synchronized stimulation signals.

The physical stimulation generator includes at least one envelope module that generates signals modulated with envelope functions with defined levels and periods. Typically the oscillator is in free-running mode and transmits the stimulation signal to the envelope module. The envelope module modulates the amplitude of the stimulation signal and is usually triggered by the communication protocol or control signal. When triggered the envelope module outputs the oscillator signal at a defined course of gain for a defined period. Such the full course of the envelope creates a train of stimulation pulses with levels following the envelope as shown in FIG. 6. Course and period parameters are typically adjustable and controlled by the communication protocol or a control signal. This is essential for live stimulation signals and communication applications. In a preferred embodiment the initial level, the attack period, the peak level, a decay period, a hold level, a minimum hold period and a release period is modulated by a communication protocol e.g. MIDI or control signals. The initial level allows rapid stimulation e.g. to exceed the threshold for muscle stimulation, the peak level achieves dynamic stimulation and the hold level equals preferably the permanent gain and adjusts a comfortable level at least for the minimum hold period or as long as the trigger signal e.g the key stroke of a keyboard controller continues. Then the during the release period the signal fades out to the initial level or to zero level again. The most simple embodiment of the envelop module is fader for the intensity of the stimulation.

The physical stimulation generator includes at least one routing/mixer module that assigns stimulation data, communication protocol data, control data between the functional modules e.g. the oscillator, digital signal processing module, derivation module and the safety module at the output. Such the routing/mixer module configures the stimulator and is typically controlled by the digital signal processing module or directly by the communication protocol. The most simplest routing embodiment is a on/off switch. The mixer section of the routing/mixer module in addition assigns and adjusts gain and intensity. Such the levels and modulation of the parameters of the stimulation data is configured and defined. The most simplest routing embodiment is the adjustment of the level of the stimulation. Where beneficial e.g. for research and development of new applications, a modular concept with flexible routing and mixing of functional modules e.g. pulse generators, envelopes, oscillators, signal processing and modulation modules accomplish very flexible signal processing and functions.

FIG. 5 shows the routing/mixer module of the 2-channel universal stimulator. Levels and periods of the biphasic pulse oscillator are modulated for both channels either with the same intensity or switchable with inverted characteristic. Such a control signal e.g. a sine low frequency oscillator may modulate and increase the time delay for stimulator 1 with a factor defined by the mixer while in parallel the time delay for stimulator 2 is decreased with the same factor. In addition the levels and periods of the envelope and overall levels are controlled by the mixer. The routing section assigns the pitch represented by the keys, the Touch strength and velocity of the keystroke, and further the modulation wheel and the bender of the controller through the mixer to the parameters of stimulator 1 and stimulator 2. Typically the wheel defines the period of a low frequency generator that modulates either pulse levels or pulse delay or both. The pressed key represents the pitch of a musical instrument and typically defines the periods of the pulse oscillator or the envelope or both. The bender provides an offset to the pitch of the pressed key and typically modulates the periods or delay of the biphasic pulse oscillator or the period of the envelope. The touch strength typically modulates the overall stimulation level through the mixer, optional with inverted characteristics for stimulator 1 and stimulator 2. The velocity modulates peak level or attack time of the envelope.

At least one derivation 18 module is implemented for embodiments where stimulation data is generated from an input signal. The derivation 18 module analysis data representing the input signal and processes a defined transfer function e.g. arithmetic functions, translation tables or other means of digital signal processing and either generates stimulation data or modulates properties of stimulation data. Typically audio signals are transferred to non auditory stimulation data. Such a derivation module may generate either electrical or vibrational or both and mimics and support music reception e.g. for deaf individuals. Typical characteristics of music e.g level, pitch, chords, rhythm are analysed and extracted in the time or frequency domain. For speech typically level, envelope, vowels and consonants are analysed. The derivation module 18 generates or modulates the stimulation signal through the routing/mixer module 16 and is controlled and exchanges data through the digital signal processing module. For those skilled in the art various implementation, variants and methods are apparent. One embodiment provides plug-in function, such that the derivation module is a specialized third party SW module. In another embodiment the derivation module is partially implemented in hardware, e.g. ASIC.

The safety module monitors stimulation signals permanently and if a critical parameter occurs e.g. DC content, overstimulation or high electrode resistance than the safety circuit takes immediate action and shuts off or limits the waveforms to prevent from potentially overstimulation or other harmful physical reactions. The limits are set to national regulatory requirements. The safety circuit needs to operate independently and shall reside as close near the input or output as possible. Optional and preferably a safety module is implemented at the output of the physical stimulation generator module to process and warrant safe and consistent stimulation data and therapies. In addition to prevent wrong stimulation signals or data to be routed to a stimulation interface, a digital right management system DRM may authorize the correct use. DRM standards may be adopted to match requirements for medical applications. Furthermore the safety module may be configured to process and warrant user comfortable stimulation levels for the user.

The physical stimulation player module is typically embodied in a separate housing such that it receives data defining the stimulation and processes and outputs stimulation data at least to one physical stimulation interface. Key element of the invention is to separate the generation and the playback function. While both modules may be embodied in the same housing and utilize the same computer system the playback function is supposed to be typically performed offline and remote within the majority of future embodiments. The player module typically performs functions as known from multimedia player e.g. selection of therapy data, adjustment of comfortable levels, playback, pause, stop, wind and rewind and processing of the signal. In addition data transfer, decoding and storage function may be implemented. In a typical embodiment the playback function comprises the local stored data or streamed data by a wired or wireless interface, transferred from a computer or provider service optional with decoding and with digital right management. The player may operate as remote controlled black box but typically contains a graphical display and human interface.

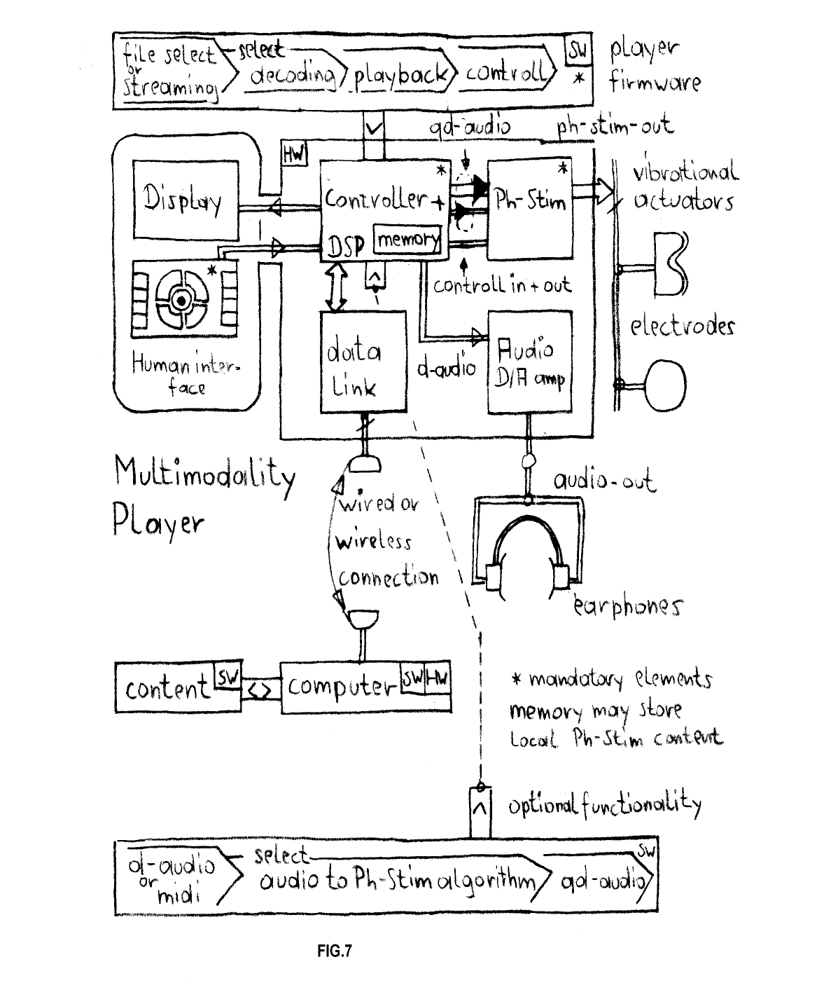

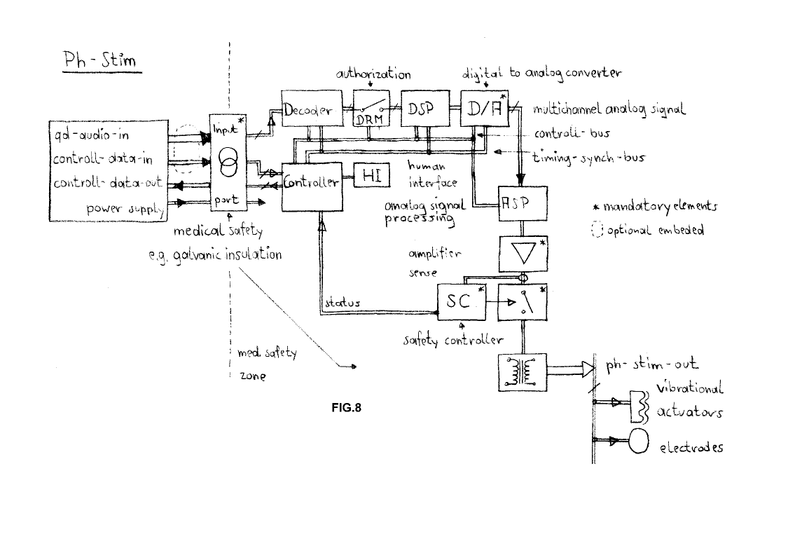

FIG. 7 shows a typical setup of a portable combined multimedia and stimulation player. The minimum configuration is build of a controller with memory, Physical stimulation interface module, human interface and a player firmware and is embodied in a battery powered small housing assembly. The controller processes the synchronized workflow, storage, buffering of data, decoding and playback of stored and streamed combined stimulation. Display and keypad are typical components of the human interface. The Physical stimulation interface transfers the decoded digital physical stimulation into analog stimulation signals that drive the actuators e.g. electrodes and vibrational actuators. Physical stimulation interface functions are typically highly integrated and merged into one controller that provides all portable combined stimulation functions. While not mandatory a data link is typically implemented. The controller downloads offline and streams online through the data link combined stimulation from a computer or a file server network e.g. internet services. Data e.g. combined stimulation or player software and firmware may be transferred to the controller via a wired link e.g. USB or wireless link e.g. Bluetooth. The data link contains safety provisions for medical safety for the applicant e.g. galvanic insulation. In addition or alternatively depending on the stimulation modality and the regulatory requirements thereof the output signal obtains galvanic insulation e.g. by an isolation transformer. Advanced configurations may include a digital to analog converter D/A with analog audio output, decoder, digital right management DRM or alternative authorization methods, digital signal processor and analog signal processing for signal conditioning. The combined stimulation player is operated by the human interface. A remote controller may be implemented for user convenience. The combined stimulation player may be designed single channel, multichannel and mixed modality including audio. In another embodiment the player contains in addition a derivation module. Typically the derivation module generates, modulates or controls in realtime audio to electrical or vibrational stimulation or both.

Physical interfaces perform the transformation of actual physical input or physical stimulation output is provided by highly specialized interfaces that contain signal conditioning including galvanic insulation when needed and safety circuits specifically designed for the specific characteristics of the modality, the connected transducer and the involved part of the body. The physical interface complies with the standards, design principles and testing requirements of medical device directives and masters the safety and regulatory challenges introduced by the connected non medical devices by hard wired or software programmed function modules. Such physical interfaces generate or read typically digital audio data representing the stimulation and optional control data. Digital rights management may be incorporated to label authorize and route the appropriate data to the corresponding application.

The disclosed stimulator device contains at least one stimulation interface which electrically drives at least one transducer that stimulates a body. In addition typically one transducer stimulates the auditory function. At least one transducer may stimulate the sensory function by a mechanical actuator such that defined regions of the body are stimulated. Alternatively or in combination at least one transducer may stimulate the sensory function by electrodes such that action potentials of nerve fibers at defined regions of the body are innervated. At least one transducer may stimulate the motor function by electrodes such that defined muscles contract. The physical stimulation interface (Ph-Stim) converts digital data presenting the analog stimulation signal provided by the playback process into physical signals and amplifies and conditions it to meet the physical and biological characteristics of each targeted auditor, sensory or motor function. The Ph-Stim drives the actuators e.g. electrodes, piezo- , electromagnetic or magnetorestrictive actuators. For quasi realtime processes the latency needs to be a defined constant and compensated between different modalities.

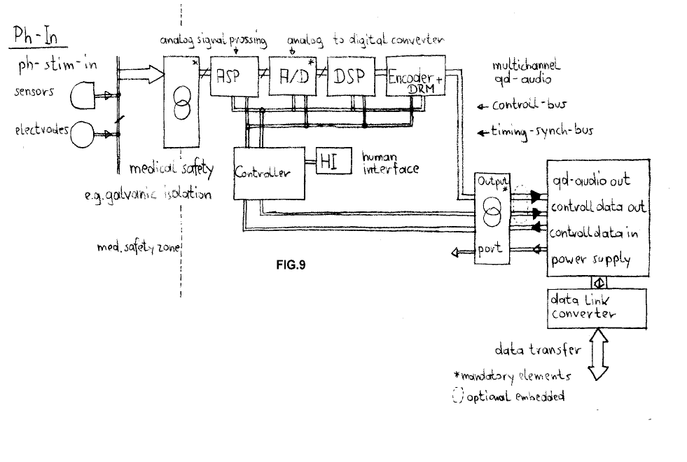

FIG. 8 shows a block diagram of a typical physical stimulation interface (Ph-Stim). The minimum configuration of a Ph-Stim is build of an input port, a digital to analog converter D/A, an amplifier stage and a safety controller with a switch and limiter for the output. The input port receives the digital data representing the physical stimulation converts and processes it typically in an digital audio format, receives optional control data either embodied or by an extra channel e.g. for configuration, identification etc. and optional outputs control-data e.g. for status information. Data may be buffered and transferred to the import port via a wired link e.g. USB or wireless link e.g. Bluetooth. The input port provides medical safety provisions for the applicant e.g. by galvanic insulation. In addition or alternatively depending on the stimulation type and the regulatory requirements thereof the output signal obtains galvanic insulation e.g. by an isolation transformer. Galvanic insulation of external power supply provided by wires is mandatory for most physical stimulation modalities. Alternatively internal power supply e.g. batteries may be implemented. The digital to analog converter D/A converts the digital data representing the physical signal into the analog waveform. The amplifier amplifies and conditions the analog signal for the corresponding transducer and the properties of the stimulated part of the body and application. The safety controller senses the output and optional feedback signal directly from the transducers and compares safety relevant signal properties e.g. level, dose, slew rate according to the regulatory safety requirements and comfortable settings and activates a shut down switch and limiter when safe and comfortable area is exceeded. Advanced configurations may include a decoder, digital right management DRM or alternative authorization methods, a digital signal processor and analog signal processing for remote and user controlled signal conditioning and a controller. The controller provides the synchronized workflow of the modules according to the control data and human interface and storage and buffering of data. The Ph-Stim functions may be implemented fully hard wired or may contain software and firmware. The modules may be physically highly integrated into electronic circuits. The Ph-Stim may function as a black box or, may be operated external by the control data or may contain a human interface, typically integrated switches and display. A remote controller may be implemented for user convenience. The PH-Stim module may be designed single channel, multichannel and mixed type and may be implemented as a discrete unit e.g. board, module with housing or may be embedded as functional module in a printed circuit board PCB.

For the recording of the physical data, modality specific input devices are utilized that are designed to sample physical signals from the body, provided by sensors and to convert it typically into a digital audio data format, preferable a standardized and time synchronizable digital data format.

The input device is specialised for the specific physical characteristics of the signal e.g. EMG, ECG, skin resistance, respiration. For special purposes e.g. feedback, the original analog stimulation signal for the body may be recorded the same way. Typically preamplification and signal processing of the raw information takes place to suppress or to reduce artifacts and to adopt the original signal to the characteristics of the following digital computing and transfer processes. Data may be buffered and transferred to the digital recording process via a wired port e.g. USB or wireless link e.g. Bluetooth. The data format of the Ph-In is standardized and readable for the subsequent recording process. For time critical real time processes the transfer time (latency) from the sensor to the recording process needs to be constant and defined and may not exceed a given delay. The Ph-In may receive a master time code from the recording process or generate such by a separate data link or may embed it typically into a digital audio data format. This allows for time accurate processing with the other combined signals.

FIG. 9 shows a typical block diagram of a physical input interface (Ph-In). The minimum configuration of a physical input interface Ph-In is a input port, a analog to digital converter A/D and a outport port. The input port receives the analog data provided by physical sensors e.g. electrodes. The input port may provide medical safety for the applicant e.g. by galvanic insulation. In addition or alternatively depending on the stimulation modality and the regulatory requirements thereof the output signal obtains galvanic insulation e.g. by an isolation transformer. The output port transfers the digital data representing the physical stimulation, typically a digital audio format, transfers optional control data either embodied or by an extra channel e.g. for identification, verification, synchronisation etc. and optional receives control-data e.g. for configuration, synchronisation etc. Data may be buffered and transferred to the next processing stage via a wired link e.g. USB or wireless link e.g. Bluetooth. Galvanic insulation of external power supply provided by wires is mandatory for most measured physical modalities. Alternatively internal power supply e.g. battery may be implemented. The analog to digital converter A/D converts the analog signal representing the physical signal into digital data and encodes it typically to a digital audio format. Advanced configurations may include analog signal processing with preamplification and digital signal processing for remote and user controlled signal conditioning, encoder, digital right management DRM or other method to identify and flag the verified physical signal and a controller. The controller provides the synchronized workflow of the modules according to the control data and human interface and storage and buffering of data. The analog signal processing module and digital signal processing module conditions the physical signal, detect and filter artifacts. The Ph-Stim functions may be implemented fully hard wired or may contain software and firmware. The modules may be physically highly integrated into electronic circuits. The Ph-Stim may function as a black box or, may be operated external by the control data or may contain a human interface, typically integrated switches and display. A remote controller may be implemented for user convenience. The Ph-In module may be designed single channel, multichannel and mixed modality and may be implemented as a discrete unit e.g. board, module with housing or may be embedded as functional module in a printed circuit board PCB.

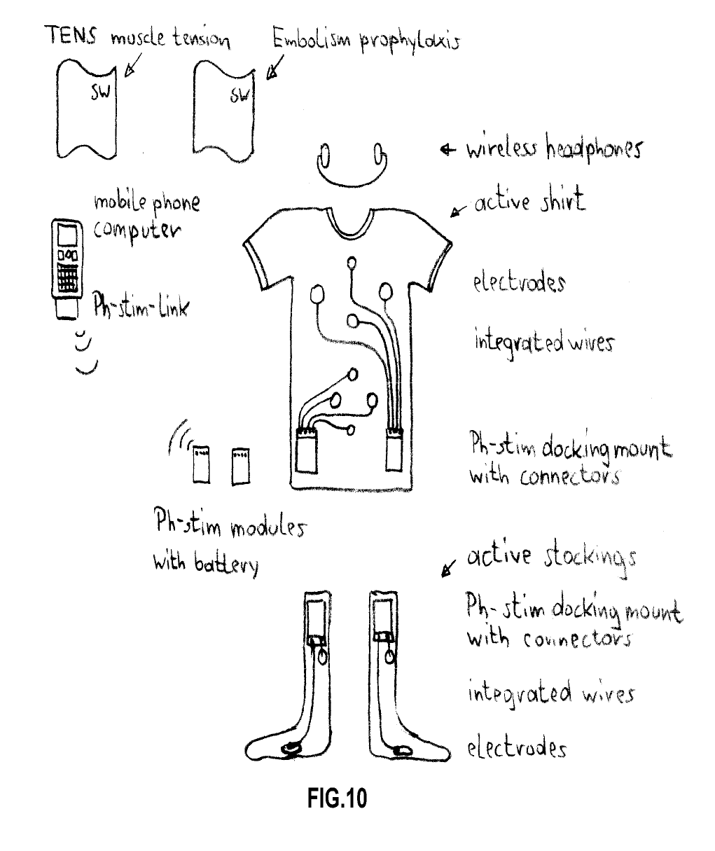

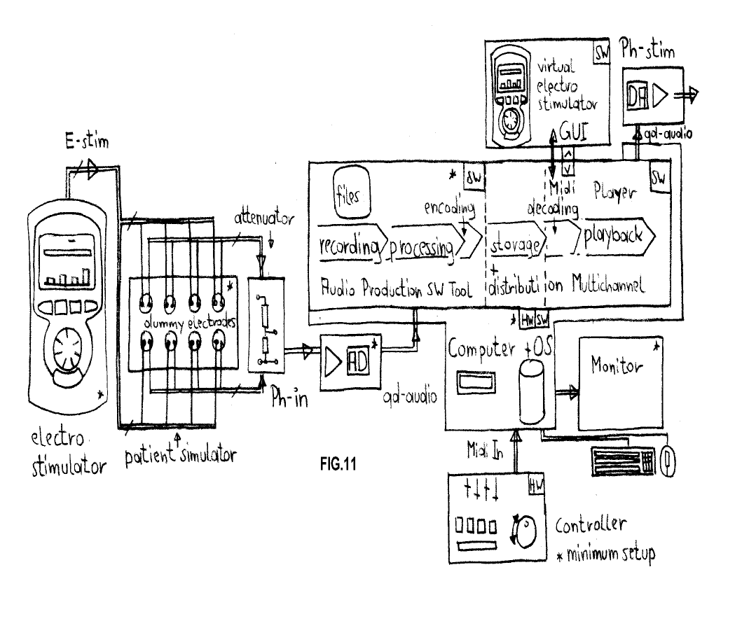

FIG. 10 shows an application specific embodiment for combined music, electrical- and vibrational stimulation. The embodiment is capable to provide multiple e.g. a) neck and back-muscle relaxation e.g. for computer workers and b) embolism prophylaxis for the lower extremities e.g. during intercontinental flights. The hardware components typically are a) a mobile phone, b) bluetooth headphones, c) a battery powered multi-channel electrical stimulator interface with radio link, d) an active shirt with a pocket for the stimulator, connector, garment integrated wires for the electrode pads and anatomically placed in neck and back region, e) a pair of active stockings with in garment integrated wired electrodes. The software for neck-back-muscle relaxation consists of a electrical stimulation software application e.g. EMS, TENS with music and matched electrical stimulation applied through by the active shirt and designed to remove muscle tension and back-pain. New special combined physiotherapy stimulation or autogenic training may be downloaded by a remote provider service with additional optional trainer interaction. In an addition embodiment a integrated derivation module generates simultaneously electrical stimulation from audio. The software for embolism prophylaxis generates electrostimulation by wired electrodes that are integrated in the stockings and promotes blood circulation and thrombosis prophylaxis e.g. during long flight periods. The stimulation setup shown in picture FIG. 10 is hosted and operated by a mobile phone. The stimulation software generate the digital physical stimulation signal transfers stimulation data preferable by wireless or wired link to the physical stimulation interface modules (Ph-Stim). The Ph-Stim modules are located near the application place of the body. In a typical embodiment the Ph-Stim output is routed by a docking mount to the integrated electrode wires of the active shirt or the active stockings. The electrodes connected to the integrated wires provide the stimulation of the respective part of the body. Alternatively other actuators e.g. vibrational actuators may be embodied to provide stimulation. Optional audio e.g. music is applied synchronously by headphones or speakers.

FIG. 11. shows a virtual physical stimulator device that samples, mimics and emulates an existing hardware stimulator. The virtual stimulator is a player for the playback of stored electrical stimulation waveforms of a real hardware stimulator. Preferentially the graphical user interface GUI displays a graphical representation of the actual hardware stimulator including the graphical representation of the operation controls which are virtually operated by keyboard, mouse or MIDI controller. In addition the GUI mimics the display of the real hardware and displays the actual settings. The operation workflow of the real hardware device is implemented by the software player. The Ph-Stim module is designed with sufficient headroom to output same electrical signal as the real hardware stimulator. The sampling of the stimulation output of the hardware stimulator is performed by stimulating a dummy probe. The analog to digital converter AD transfers the sampled data to the computer and the recording process creates a discrete file representing the waveform and the course of the real hardware stimulator. Such stimulation recordings may be stored, processed and replayed by the player on a PC or a portable device. Such virtual replica may replace the existing hardware devices. In addition the stimulation recordings and course may be postprocessed e.g. modified, modulated and combined with other stimulation waveforms and sequences.

Generator, player, physical stimulation interface and other modules may be embodied in separate housings, implemented in either hardware or software or a combination thereof and integrated in separate computer systems but are typically connected online or offline by data transfer and are technically addicted to form a functioning system. The invention is typically implemented as combination of software and hardware (e.g., a computer program product) and hardware. Still embodiments entirely in hardware, or entirely software (e.g., a computer program product) with the exception at least of the output stages of the physical interfaces are feasible embodiments.

The present invention may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof.

Computer program logic implementing all or part of the functionality previously described herein may be embodied in various forms, including, but in no way limited to, a source code form, a computer executable form, and various intermediate forms (e.g., forms generated by an assembler, compiler, linker, or locator.) Source code may include a series of computer program instructions implemented in any of various programming languages (e.g., an object code, an assembly language, a high-level language such as C, C++, JAVA, or HTML, MATLAB or a visual programming language such as LABVIEW) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies, networking technologies, and internetworking technologies. The computer program may be distributed in any form as a removable storage medium with accompanying printed or electronic documentation (e.g., shrink wrapped software or a magnetic tape), preloaded with a computer system (e.g., on system ROM or fixed disk), or distributed from a server or electronic bulletin board over the communication system (e.g., the Internet or World Wide Web.)

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL, AHDL, Verilog), or a PLD programming language (e.g., PALASM, ABEL, or CUPL.)

Although various exemplary embodiments of the invention have been disclosed, it should be apparent to those skilled in the art that various changes and modifications can be made which will achieve some of the advantages of the invention without departing from the true scope of the invention.

### Reference To Deposited Biological Material

not applicable

### Sequence Listing Free Text

not applicable

What is claimed is:

1. A stimulator device for stimulation of sensory and motor functions, the device comprising:
a physical stimulation generator module
a physical stimulation player module
a physical stimulation interface that electrically drives at least one transducer that stimulates a body
a digital signal processor at least for the generation of complex electrical stimulation waveforms with interactive ability to change multiple processing parameters immediately and simultaneously
a communication protocol that enables at least the physical stimulation generator module to communicate control and synchronize the processing of stimulation data
a user safety monitoring and limiter function configurable to comply with regulatory requirements

2. A device according to claim 1,
wherein the physical stimulation generator includes at least one oscillator module that generates biphasic pulses with defined levels, periods and repetition rate.

3. A device according to claim 1,
wherein the physical stimulation generator includes at least one oscillator modules that generates waveforms based on algorithms including sine, triangle, trapezoid, pulse, triangle, ramp, white- and pink noise.

4. A device according to claim 1,
wherein the physical stimulation generator includes at least one sample player module that generates waveforms represented by data samples with defined period and repetition rate.

5. A device according to claim 1,
wherein the physical stimulation generator includes at least one envelope module that generates signals modulated with envelope functions with defined levels and periods.

6. A device according to claim 5,
wherein the envelope function is defined by start-, peak-, hold-, release level with and corresponding attack-, decay-, minimum hold- and release periods.

7. A device according to claim 1,
wherein the physical stimulation generator includes at least one mixer module such that parameters and routing of stimulation data is controlled with defined gain by at least one control signal.

8. A device according to claim 1,
wherein the physical stimulation generator includes at least one derivation module such that stimulation data is generated from an input signal by analysis and defined transfer function.

9. A device according to claim 8,
wherein characteristic properties of an audio input signal are analysed and transferred to non auditory stimulation data.

10. A device according to claim 1,
wherein characteristic properties of an audio input signal are analysed and transferred to change and modulate properties of non auditory stimulation data.

11. A device according to claim 1,
wherein the physical stimulation player module is embodied in a separate housing such that it receives data defining the stimulation and processes and outputs stimulation data at least to one physical stimulation interface.

12. A device according to claim 1,
where a musical instrument type controller controls at least the physical stimulation generator module by the communication protocol.

13. A device according to claim 1,
where an advanced graphical user interface immediately displays function, operation and configuration parameters such that a virtual representation of a hardware embodied device is given.

14. A device according to claim 1,
wherein at least one transducer stimulates the auditory function.

15. A device according to claim 1,
wherein at least one transducer stimulates the sensory function by a mechanical actuator such that defined regions of the body are stimulated to vibrate.

16. A device according to claim 1,
wherein at least one transducer stimulates the sensory function by electrodes such that action potentials of nerve fibers at defined regions of the body are innervated.

17. A device according to claim 1,
wherein at least one transducer stimulates the motor function by electrodes such that defined muscles contract.

18. A method of producing stimulation of sensory and motor functions, the method comprising:
generating complex electrical stimulation waveforms with interactive ability to change multiple processing parameters immediately and simultaneously by digital signal processing and communicating, controlling and synchronizing at least the processing of stimulation data by a communication protocol and monitoring and limiting stimulation signals to comply with regulatory requirements.

20. A computer program product in a computer readable storage medium, the product including
program code for producing a data signal for the stimulation of sensory and
motor functions, the product comprising:
program code for generating a physical stimulation generator function
program code for generating a physical stimulation player function
program code for generating a device driver for an physical stimulation interface program code for digital signal processing at least the generation of complex electrical stimulation waveforms with interactive ability to change multiple processing parameters immediately and simultaneously
program code for a communication protocol that enables at least the physical stimulation generator module to communicate, control and synchronize the processing of stimulation data
and program code for a user safety monitoring and limiter function configurable to comply with regulatory requirements.
